(19) United States
(12) Patent Application Publication
(10) Pub. No.: US 2020/0110073 A1
(43) Pub. Date: Apr. 9, 2020

(54) RENAL DISEASE TARGETS

(71) Applicant: The Jackson Laboratory, Bar Harbor, ME (US)

(72) Inventor: Ron Korstanje, Bar Harbor, ME (US)

(73) Assignee: The Jackson Laboratory, Bar Harbor, ME (US)

(21) Appl. No.: 16/591,278

(22) Filed: Oct. 2, 2019

Related U.S. Application Data
(60) Provisional application No. 62/741,363, filed on Oct. 4, 2018.

(51) Int. Cl. G01N 33/50; C12N 5/071; C12Q 1/02; C12Q 1/6851; C12Q 1/6827

(52) U.S. Cl. CPC G01N 33/5023; G01N 33/5044; C12N 5/0686; C12Q 2600/158; C12Q 1/6851; C12Q 1/6827; C12Q 1/025

(57) ABSTRACT

Provided herein, in some embodiments, are methods for modifying expression and/or activity of genes associated with renal disease. Also provided herein are methods for identifying agents that modify expression and/or activity of genes associated with renal disease.

### RELATED APPLICATION

This application claims the benefit under 35 U.S.C. § 119(e) of U.S. provisional application No. 62/741,363, filed Oct. 4, 2019, which is incorporated by reference herein in its entirety.

### FEDERALLY SPONSORED RESEARCH

This invention was made with government support under Grant No. DK076169 awarded by National Institute of Diabetes and Digestive Kidney Disease (NIDDK) Diabetic Complications Consortium and Grant No. P30CA034196 awarded by National Cancer Institute. The government has certain rights in the invention.

### BACKGROUND

The prevalence of chronic kidney disease (CKD) is rising worldwide, and currently 10-15% of the global population suffer from CKD and its devastating complications¹. Although the adult human kidney has some ability to recover from acute kidney injury through cellular proliferation of the damaged intrarenal tissues, regenerating nephrons through de novo nephron development is considered unlikely, as the formation of new nephrons in humans is terminated at the embryonic stages.

### SUMMARY

The present disclosure provides, in some aspects, methods of identifying an agent that modifies expression of a renal disease target. The methods, in some embodiments, comprise contacting a kidney cell with an agent of interest, and assaying for expression of a gene or activity of a product described herein as being differentially expressed before and after hibernation of a black bear or tenrec (see, e.g., Table 1). In other aspects, the present disclosure provides methods of treating a subject having a renal disease, comprising delivering to the subject an agent that modifies expression of a gene or activity of a product described herein as being differentially expressed before and after hibernation of a black bear or tenrec.

Like some fish species, members of the bear family (Ursidae) may undergo renal regeneration through nephron neogenesis in the event of renal injury. Understanding how members of the bear family deal with periods of decreased kidney function during hibernation (biomimicry), for example, may provide a more thorough understanding of kidney disease and may enable the development of treatments.

The American black bear hibernates for up to seven months annually. During this period, black bears do not eat, drink, urinate, or defecate. Bear hibernation is a state similar to prolonged sleep during which body temperature is reduced by 1-8° C.³, there is a 20-50% reduction in metabolic rate with a depressed heart rate⁴, and the volume of urine produced is reduced by 95%⁵. The small volumes of urine and urea that enter the bladder during hibernation are reabsorbed across the bladder epithelium⁵, and the urea is recycled for production of new protein⁶. Throughout hibernation the kidney continues to concentrate urine and produces renin⁷, erythropoietin⁵, and vitamin D 1-α-hydroxylase⁸. Hibernating bears have the ability to prevent azotemia (high levels of nitrogen-containing compounds in the blood, common in human patients with renal function), but the mechanism is unknown.

Understanding these processes in the American black bear may lead to novel therapies for treating human conditions related to resistance to the complications of chronic kidney disease (CKD) and recovery from acute kidney injury. It is likely that unique kidney features in the American black bear allow them to endure lower functioning during hibernation and recovery soon after hibernation. These unique kidney features are likely in part encoded in the genome sequence and gene expression patterns of the bear. To address this, the present disclosure provides data from a high-throughput sequencing analysis of genomic DNA and RNA isolated from kidneys of wild American black bears. A de novo assembly and annotation of the complete genome was generated, transcription profiles of kidneys collected in the fall (before hibernation) and in the spring (within weeks after hibernation) were compared and analyzed.

A similar sequencing analysis was performed using kidneys obtained from tenrecs, a hibernating species Afrotherian family Tenrecidae endemic to Madagascar. As a result of convergent evolution, some tenrecs resemble hedgehogs, shrews, opossums, or mice.

Thus, provided herein, in some aspects are methods that comprise contacting a kidney cell with an agent that modifies (a) expression of a gene selected from any one of the genes listed in Table 1 (shown to exhibit differential expression), or (b) activity of a product encoded by a gene selected from any one of the genes listed in Table 1.

Other aspects of the present disclosure provide methods that comprise delivering to a subject having a renal disease an agent that modifies (a) expression of a gene selected from any one of the genes listed in Table 1, or (b) activity of a product encoded by a gene selected from any one of the genes listed in Table 1.

In some embodiments, the agent modifies (a) expression of a gene selected from ACSL5, CISH, SLCO1C1, SERPINC1, PLCXD2, PLCD3, ABCC3, MAST3, TSPOAP1, CCM2L, MAP3K12, PRKD2, SEPW1, SHANK3, RTN4RL2, COL11A2, FAM81A, FAM83H, SLC46A1, MEGF8, POMT1, AATK, CLEC18A, MYO7B, SLC5A10, GZMA, API5, CTH, TMEM27, CA7, ABRACL, PTMA, PPDPF, SRI, MCU, HIPK3, GCHFR, CREM, PTP4A1, TST, IER3, GSKIP, SOCS2, C3ORF18, NDST3, LYPLA1, CSMD1, MHC, ENSCAFG00000002590, CGNL1, TDRD5, RAB38 and homologs thereof. For example, the agent may modify (a) expression of a gene selected from NDST3, CISH, SLCO1C1, SOCS2, SERPINC1, CGNL1, TDRD5, and RTN4RL2, or (b) activity of a product encoded by a gene selected from NDST3, CISH, SLCO1C1 , SOCS2, and SERPINC1, CGNL1, TDRD5, and RTN4RL2. In some embodiments, the agent increases (a) expression of a gene selected from NDST3, CISH, SLCO1C1, SOCS2, TDRD5, and SERPINC1, or (b) activity of a product encoded by a gene selected from NDST3, CISH, SLCO1C1, SOCS2, TDRD5, and SERPINC1. For example, the agent may increase (a) expression of NDST3, or (b) activity of a product encoded by NDST3. As another example, the agent may increase (a) expression of CISH, or (b) activity of a product encoded by CISH. As yet another example, the agent may increase (a) expression of SLCO1C1, or (b) activity of a product encoded by SLCO1C1. As still another example, the agent may increase (a) expression of SOCS2, or (b) activity of a product encoded by SOCS2. As another example, the agent may increase (a) expression of TDRD5, or (b) activity of a product encoded by TDRD5. As a further example, the agent may increase (a) expression of SERPINC1, or (b) activity of a product encoded by SERPINC1.

In some embodiments, the expression or the activity of any one or more of the foregoing genes is increased by at least 1.5 fold or by at least 2 fold.

In some embodiments, the agent decreases (a) expression of RTN4RL2, or (b) activity of a product encoded by RTN4RL2. In other embodiments, the agent decreases (a) expression of CGNL1, or (b) activity of a product encoded by CGNL1. For example, the expression or the activity may be decreased by at least 1.5 fold.

In some embodiments, the kidney cell is a black bear kidney, a mouse kidney cell, a tenrec kidney cell, or a human kidney cell. In some embodiments, the kidney cell is a human kidney cell.

In some embodiments, a subject is a rodent, such as a mouse. In some embodiments, the subject is a human.

Other aspects of the present disclosure provide methods that comprise contacting a kidney cell with an agent that modifies expression of, or modifies activity of a product encoded by, a pathway gene upstream from a gene selected from NDST3, CISH, SLCO1C1, SOCS2, SERPINC1, CGNL1, and TDRD5.

Still other aspects of the present disclosure provide methods that comprise contacting a kidney cell with an agent that modifies expression of, or modifies activity of a product encoded by, a pathway gene downstream from a gene selected from NDST3, CISH, SLCO1C1, SOCS2, SERPINC1, CGNL1, and TDRD5.

### BRIEF DESCRIPTION OF THE DRAWINGS

FIGS. 1A-1C: Sequencing depth and comparison of several characteristics of the black bear, panda, and polar bear genome assemblies. (FIG. 1A) Distribution of sequencing depth of the assembled genome. (FIG. 1B) Completeness and contiguity of the assembly was estimated using CEGMA (Core Eukaryotic Genes Mapping Approach) by screening against 248 universal eukaryotic single-copy genes. Results for the black bear (85.5% completeness) were comparable to the panda (88.7%) and polar bear (90.7%). (FIG. 1C) Density plot of the % GC content in the bear genomes comparing the black bear (solid line) with the panda (dashed line) and polar bear (dotted line). The narrower distribution in the black bear might indicate fewer repetitive sequences.

FIG. 2: Comparison of different types of repeats between the black bear and several mammalian species shows a lower number of repeat sequences in the black bear.

FIG. 3: Volcano plot of the differential gene expression between spring and fall with CISH, SLCO1C1, and NDST3 being upregulated >2-fold in the spring compared to the fall. All genes (169) with an adjusted P-value below 0.05 are indicated in gray, and include RTN4RL2, SERPINC1, and SOCS2.

FIG. 4: Cluster analysis of the gene expression profiles showing the fifty (50) genes with the lowest P-value.

### DETAILED DESCRIPTION

The prevalence of chronic kidney disease (CKD) is rising worldwide and 10-15% of the global population currently suffers from CKD and its complications. Given the increasing prevalence of CKD there is an urgent need to find novel treatment options. The American black bear (Ursus americanus) copes with months of lowered kidney function and metabolism during hibernation without the devastating effects on metabolism and other consequences observed in humans. In a biomimetic approach to better understand kidney adaptations and physiology in hibernating black bears, a high-quality genome assembly was established. Subsequent RNA-Seq analysis of kidneys comparing gene expression profiles in black bears entering (late fall) and emerging (early spring) from hibernation identified 169 protein-coding genes that were differentially expressed. Of these, 101 genes were downregulated and 68 genes were upregulated after hibernation. Fold changes ranged from 1.8-fold downregulation (RTN4RL2) to 2.4-fold upregulation (CISH). Most notable was the upregulation of cytokine suppression genes (SOCS2, CISH, and SERPINC1) and the lack of increased expression of cytokines and genes involved in inflammation. A similar study was performed with tenrecs. Two genes were present in both the black bear and tenrec datasets: CGNL1 and TDRD5. CGNL1 was downregulated after hibernation, while TDRD5 was upregulated after hibernation. The identification of these differences in gene expression in the black bear and tenrec kidney may provide new insights in the prevention and treatment of CKD.

In some aspects, the present disclosure provides methods of contacting a kidney cell with an agent that modifies the expression of a gene or the activity of a product of a gene identified herein as differentially expressed (pre-hibernation v. post-hibernation) in kidneys of black bears and/or tenrecs. For example, the methods provided herein may be used to identify an agent that modifies the expression of a gene or the activity of a product of a gene identified herein as differentially expressed in kidneys of black bears and/or tenrecs. In some embodiments, the methods comprise assaying for a change in expression of a gene (e.g., level of mRNA and/or protein) and/or activity of a product encoded by a gene, relative to a control or relative to baseline. For example, a change may be an increased level of expression of the gene and/or activity of a product encoded by a gene (e.g., by at least 0.5-fold, at least 1-fold, at least 1.5-fold, at least 2.0-fold, at least 2.5-fold, at least 3-fold, at least 3.5-fold, at least 4.0-fold, at least 4.5-fold, or at least 5-fold), or a decreased level of expression of the gene and/or activity of a product encoded by a gene (e.g., by at least 0.5-fold, at least 1-fold, at least 1.5-fold, at least 2.0-fold, at least 2.5-fold, at least 3-fold, at least 3.5-fold, at least 4.0-fold, at least 4.5-fold, or at least 5-fold). In some embodiments, the methods further comprise identifying the agent of interest as a candidate therapeutic agent for treating a renal disease if the change in expression and/or activity is at least at least 0.5-fold, at least 1-fold, at least 1.5-fold, at least 2.0-fold, at least 2.5-fold, at least 3-fold, at least 3.5-fold, at least 4.0-fold, at least 4.5-fold, or at least 5-fold, relative to a control or relative to baseline. For example, the methods may further comprise identifying the agent of interest as a candidate therapeutic agent for treating a renal disease if the change in expression and/or activity is 1.5-fold to 3-fold, relative to a control or relative to baseline. One skilled in the art understands how to perform a controlled analysis; thus, the terms “control” and “baseline” can be easily determined by one skilled in the art. The control may be, for example, expression of the gene and/or activity of a product encoded by a gene assayed following contacting a similar kidney cell with an inert substance (e.g., buffer only). Baseline may be, for example, the expression level of a gene or activity level of a gene product assayed prior to contacting a kidney cell with an agent of interest (e.g., one day to one month prior). An agent, in some embodiments, is a therapeutic agent and/or a prophylactic agent. An agent may be a biomolecule or a chemical agent. In some embodiments, an agent is a polynucleotide (e.g., double-stranded or single-stranded DNA or RNA, such as a guide RNA (gRNA) (e.g., in combination with Cas9), messenger RNA (mRNA), or an RNA interference (RNAi) molecule, such as antisense RNA, small interfering RNAs (siRNAs), short hairpin RNAs (shRNAs), and/or microRNAs (miRNAs)). In some embodiments, an agent is a polypeptide (e.g., protein and/or peptide). Non-limiting examples of polypeptides include antibodies (e.g., monoclonal antibodies and/or antibody fragments, such as single chain variable fragments (scFvs)). An agent, in some embodiments, is a cellular agent, such as a stem cell (e.g., pluripotent stem cell, such as an induced pluripotent stem cell) or immune cell (e.g., T cell or CAR-T cell). In some embodiments, an agent is small molecule drug (e.g., chemical compound).

An agent is considered to modify expression of a gene if expression of the gene is increased or decreased following exposure of the agent to a cell comprising the gene. In some embodiments, the change in gene expression is relative to a control, such as gene expression from a cell not exposed to the agent. In some embodiments, an agent increases expression of a gene by at least 10%, at least 20%, at least 30%, at least 40%, at least 50%, at least 60%, at least 70%, at least 80%, at least 90%, or at least 100% (e.g., by 10%-100%), relative to a control. In some embodiments, an agent decreases expression of a gene by at least 10%, at least 20%, at least 30%, at least 40%, at least 50%, at least 60%, at least 70%, at least 80%, at least 90%, or at least 100% (e.g., by 10%-100%), relative to a control.

Likewise, an agent is considered to modify activity of a product (e.g., protein) encoded by a gene if activity of the product is increased or decreased following exposure of the agent to a cell comprising the gene encoding the protein. In some embodiments, the change in activity is relative to a control, such as activity in a cell not exposed to the agent. In some embodiments, an agent increases activity of a product by at least 10%, at least 20%, at least 30%, at least 40%, at least 50%, at least 60%, at least 70%, at least 80%, at least 90%, or at least 100% (e.g., by 10%-100%), relative to a control. In some embodiments, an agent decreases activity of a product by at least 10%, at least 20%, at least 30%, at least 40%, at least 50%, at least 60%, at least 70%, at least 80%, at least 90%, or at least 100% (e.g., by 10%-100%), relative to a control.

In some embodiments, an agent increases expression of a gene by at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold (e.g., 1.5 fold-20-fold).

In some embodiments, an agent decreases expression of a gene by at least 1.5-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 16-fold, at least 17-fold, at least 18-fold, at least 19-fold, or at least 20-fold (e.g., 1.5 fold-20-fold).

Contacting a cell with an agent includes exposing a cell (e.g., in vivo or in vitro) to an agent (e.g., a therapeutic agent) such that the cell comes into physical contact with the agent. For example, the step of contacting a cell with an agent may include delivering the agent to a composition that includes the cell, and/or delivering the cell to a composition that includes the agent. A cell may also be contacted by an agent when the agent is delivered to a subject in which the cell is present (e.g., in blood or other tissue or organ).

Kidney cells of the present disclosure may be selected from black bear kidney cells, tenrec kidney cells, rodent kidney cells (e.g., rat kidney cells and/or mouse kidney cells), or human kidney cells. In some embodiments, kidney cells are selected from human kidney cells, such as human embryonic kidney (HEK) cells, for example, HEK293 cells. Kidney cells of the present disclosure may be used to test the function of an agent, for example, the extent to which (if any) and agent modifies expression of a gene or activity of a product encoded by a gene as provide herein. Thus, in some embodiments, kidney cells may be modified (e.g., genomically modified) to express or overexpress (e.g., knock in) or reduce (e.g., knock out) expression of any one or more of the genes (or upstream or downstream genes) as provided herein (e.g., see Table 1).

Other aspects of the present disclosure provide methods of delivering to a subject having a renal disease an agent that modifies the expression of a gene or the activity of a product of a gene identified herein as differentially expressed (pre-hibernation v. post-hibernation) in kidneys of black bears and/or tenrecs. For example, the methods provided herein may be used to treat the renal disease. The term treat, as known in the art, refers to the process of alleviating at least one symptom associated with a disease. A symptom may be a physical, mental, or pathological manifestation of a disease. Symptoms associated with renal diseases are known and include, for example, fatigue, trouble sleeping, dry and itchy skin, the need to urinate more often, blood in urine, puffiness around the eyes, and/or swollen ankles and/or feet. It should be understood that the term “treatment,” herein, encompasses prevention (prophylaxis).

Delivery of an agent to a subject may be by any route known in art. For example, delivery of the agent may be intravenous (e.g., dialysis), intramuscular, or subcutaneous. Other delivery routes may be used.

Subjects of the present disclosure may be selected from black bears, tenrecs, rodents (e.g., rats and/or mice), tenrecs, and humans. In some embodiments, the subject is a transgenic mouse that express or overexpress (e.g., knock in) or exhibits reduced (e.g., knock out) expression of any one or more of the genes (or upstream or downstream genes) as provided herein (e.g., see Table 1). In some embodiments, the subject is a human subject, for example, a subject having (e.g., diagnosed with) a renal disease.

### Renal Disease

In some aspects, the present disclosure provides a method of delivering to a subject having a renal disease an agent that modifies the expression of a gene or activity of a product of a gene. A renal disease is a disorder of the kidneys. Manifestations of renal disease include abnormal structure(s), function(s), or other process(es) in the kidneys.

The kidneys are organs in vertebrates that filter blood and produce urine by removing waste products (e.g., urea, hydrogen, ammonium, potassium, and uric acid) and reabsorbing nutrients (e.g., solute-free water, sodium, bicarbonate, glucose, and amino acids). Kidneys regulate whole-body homeostasis by managing acid-base balance, electrolyte levels, and blood pressure. Kidneys regulate acid-base balance by reabsorption and regeneration of bicarbonate from the blood and excretion of hydrogen ions and fixed acids into urine.

Renal diseases are common, with more than 200,000 cases diagnosed in the United States every year. Medications (e.g., vitamins, diuretics, bone marrow stimulations, dietary supplements) and therapies (e.g., dialysis, hemofiltration, kidney transplantation) slow the progression and alleviate the symptoms of renal diseases, but no cure exists. Non-limiting examples of renal disease that may be treated as provided herein (e.g., to alleviated/reduce symptoms of renal disease or to cure/eliminate symptoms of renal disease)) include diabetic nephropathy, glomerulonephritis, hydroephrosis, interstitial nephritis, kidney stones, kidney tumor, lupus nephritis, minimal change disease, nephrotic syndrome, kidney failure, renal artery stenosis, renovascular hypertension, chronic kidney disease, congenital hydronephrosis, congenital obstruction of the urinary tract, duplex kidneys, duplicated ureter, horseshoe kidney, nutcracker syndrome, polycystic kidney disease, renal aegenesis, renal dysplasia, unilateral small kidney, multicystic dysplastic kidney, and ureteroplevic junction obstruction.

Most renal diseases affect the nephrons and leave kidneys unable to remove wastes efficiently. Filtration of blood occurs in the nephrons, which are the structural and functional units of the kidney. The nephron filters the blood supplied to it via filtration, reabsorption of nutrients, secretion of waste products, and excretion of the filtrate out of the kidney. Nephrons are composed of numerous types of cells, all of which are classified as kidney cells. Non-limiting examples of kidney cells include specialized filtration cells (e.g., mesangial cells and podocytes), epithelial cells (e.g., tubule brush border cells, thin segment cells, thick ascending limb cells, distal tubule cells), specialized resorption cells (e.g., collecting duct principal cells, collecting duct intercalated cells), and renal interstitial cells (e.g., fibroblasts, immune cells, macrophages, dendritic cells, and perivascular cells).

Symptoms of renal disease include fatigue, high blood pressure, loss of appetite, malaise, water-electrolyte imbalance, kidney damage, abnormal heart rhythm, failure to thrive, fluid in the lungs, insufficient urine production, itching, kidney failure, severe unintentional weight loss, and/or swelling. Risk factors for developing renal disease include hypovolemia, dehydration, low fluid intake, medication (e.g., diuretics, antibiotics, lithium, nonsteroidal anti-inflammatory drugs), abnormal blood flow to or from the kidney, sepsis, rhabdomyolysis, multiple myeloma, systemic lupus erythematosus, Goodpasture syndrome, Wegener's granulomatosis, poorly controlled diabetes, and poorly controlled high blood pressure. In some embodiments, a subject of the present disclosure exhibits one or more symptoms and/or risk factors of a renal disease.

Alleviation of a renal disease refers to the process of making the symptoms of the renal disease less intense and/or more bearable. Treatment of a renal disease includes, in some embodiments, alleviating symptoms of a renal disease. In some embodiments, treatment of a renal disease includes curing the renal disease.

In some aspects, kidney cells of a subject having renal disease exhibit aberrant expression (e.g., increased or decreased expression) of a gene selected from any one of the genes listed in Table 1 compared to a subject not having a renal disease. In some embodiments, kidney cells of a subject exhibit decreased expression of a gene selected from Table 1. In some embodiments, kidney cells of a subject exhibit increased expression of a gene selected from Table 1.

In some aspects, kidney cells of a subject having renal disease exhibit aberrant activity (e.g., increased or decreased activity) of a product (e.g., protein) encoded by gene selected from any one of the genes listed in Table 1 compared to a subject not having a renal disease. In some embodiments, kidney cells of a subject exhibit decreased activity of a product encoded by a gene selected from Table 1. In some embodiments, kidney cells of a subject exhibit increased activity of a product encoded by a gene selected from Table 1.

### Genes of Interest

In some aspects, the present disclosure provides methods of delivering to a cell (e.g., kidney cell) or to a subject (e.g., having a renal disease) an agent that modifies the expression of a gene or the activity of a product encoded by a gene differentially expressed by kidney cells, as provided herein.

In some embodiments, an agent increases the expression of NDST3 or the activity of the NDST3 protein encoded by the NDST3 gene. The N-deacetylase and N-sulfotransferase 3 (NDST3) (Gene ID: 9348) gene encodes the NDST3 protein. The NDST3 protein is a member of the heparan sulfate family and is an essential bifunctional enzyme that catalyzes both the N-deacetylation and the N-sulfation of glucosamine (GlcNAc) of the glycosaminoglycan in the kidney. Thus, increased expression of NDST3 indicates an increase of N-sulfation of heparan sulfate in the kidney.

In some embodiments, an agent increases the expression of CISH or the activity of the CISH protein encoded by the CISH gene. The cytokine inducible SH2 containing protein (CISH) (Gene ID: 1154) gene encodes the CISH protein. The CISH protein belongs to the cytokine-induced STAT inhibitor (CIS), also known as suppressor of cytokine signaling (SOCS) or STAT-induced STAT inhibitor (SSI) family of proteins. CISH is induced by and negatively regulates the T cell receptor (TCR). The deletion of CISH in effector T cells promotes TCR signaling and subsequent cytokine release, proliferation, and survival.

In some embodiments, an agent increases the expression of SLCO1C1 or the activity of the SLCO1C1 protein encoded by the SLCO1C1 gene. The solute carrier organic anion transporter family member 1C1 (SLCO1C1) (Gene ID: 53919) gene encodes the SLCO1C1 protein. The SLCO1C1 protein mediates the sodium-independent high affinity transport of organic anions such as the thyroid hormones thyroxine (T4) and triiodothyronine (T3). SLCO1C1 may also play a role in regulating T4 flux into and out of the brain.

In some embodiments, an agent increases the expression of SOCS2 or the activity of the SOCS2 protein encoded by the SOCS2 gene. The suppressor of cytokine signaling 2 (SOCS2) (Gene ID: 8835) gene encodes the SOCS2 protein. The SOCS2 protein belongs to the cytokine-induced STAT inhibitor (CIS), also known as suppressor of cytokine signaling (SOCS) or STAT-induced STAT inhibitor (SSI) family of proteins. SOCS2 binds the cytoplasmic domain of insulin-like growth factor 1 receptor (IGF1R), and is thus though to be involved in regulating IGF1R-mediated cell signaling.

In some embodiments, an agent increases the expression of SERPINC1 or the activity of the antithrombin protein encoded by the SERPINC1 gene. The serpin family C member 1 (SERPINC1) (Gene ID: 462) gene encodes the antithrombin (also known as SERPINC1, antithrombin III) protein. The antithrombin protein is a serine protease inhibitor which is found in the blood stream and regulates clotting factor proteins involved in blood clotting. Antithrombin binds to thrombin and other clotting proteins, as well as heparin.

In some embodiments, an agent increases the expression of TDRD5 or the activity of the TDRD5 protein encoded by the TDRD5 gene. The tudor domain containing 5 (TDRD5) (Gene ID: 163589) gene encodes the TDRD5 protein. The TDRD5 protein interacts with Piwi proteins and may silence retrotransposons and regulate piRNAs. piRNA has been shown to be involved in regeneration in lower organisms, such as the tunicate (Rinkevich et al., Piwi-positive cells that line the vasculature epithelium, underlie whole body regeneration in a basal chordate. Dev Biol. 2010 Sep. 1; 345(1):94-104). Further, while it is thought to be testis-specific, expression in the glomeruli has been confirmed via in situ hybridization (data not shown).

In some embodiments, an agent decreases the expression of RTN4RL2 or the activity of the RTN4RL2 protein encoded by the RTN4RL2 gene. The reticulon 4 receptor like 2 (RTN4RL2) (Gene ID: 349667) gene encodes the RTN4RL2 protein. The RTN4RL2 protein is a cell surface receptor that regulates postnatal brain development by inhibiting neurite outgrowth, contributing to normal axon migration across the brain midline, and formation of the corpus callosum.

In some embodiments, an agent decreases the expression of CGNL1 or the activity of the CGNL1 protein encoded CGNL1 gene. The cingulin-like 1 (CGNL1) (Gene ID: 84952) gene encodes the CGNL1 protein, which localizes to both adherens and tight cell-cell junctions and mediates junction assembly and maintenance by regulating the activity of the small GTPases RhoA and Rac1. These processes are important in tubular function.

Agents of the present disclosure may be used to modify any one or more of the following genes or homologs thereof: ACSL5, PLCXD2, PLCD3, ABCC3, MAST3, TSPOAP1, CCM2L, MAP3K12, PRKD2, SEPW1, SHANK3, COL11A2, FAM81A, FAM83H, SLC46A1, MEGF8, POMT1, AATK, CLEC18A, MYO7B, SLC5A10, GZMA, APIS, CTH, TMEM27, CA7, ABRACL, PTMA, PPDPF, SRI, MCU, HIPK3, GCHFR, CREM, PTP4A1, TST, IER3, GSKIP, SOCS2, C3ORF18, LYPLA1, CSMD1, MHC, ENSCAFG00000002590, and/or RAB38.

The acyl-CoA synthetase long chain family member 5 (ACSL5) (Gene ID: 51703) gene encodes the ACSL5 protein. The ACSL5 protein activates long-chain fatty acids for both synthesis of cellular lipids and degradation by beta-oxidation. ACSL5 may activate fatty acids from exogenous sources for the synthesis of triacylglycerol destined for intracellular storage.

The phosphatidylinositol specific phospholipase C X domain containing 2 (PLCXD2) (Gene ID: 257068) gene encodes the PLCXD2 protein. The PLCXD2 protein belongs to a family of phosphatidylinositol (PI)-specific phospholipase C (PLC) enzymes. PLCXD2 significantly increased the basal turnover of the phosphatidylinositol pool in a calcium-independent manner.

The phospholipase C delta 3 (PLCD3) (Gene ID: 113026) gene encodes the PLCD3 protein. The PLCD3 protein hydrolyzes the phosphatidylinositol 4,5-bisphosphate (PIP2) to generate 2 second messenger molecules diacylglycerol (DAG) and inositol 1,4,5-triphosphate (IP3). PLCD3 is essential for trophoblast and placental development.

The ATP binding cassette subfamily C member 3 (ABCC3) (Gene ID: 8714) gene encodes the canalicular multispecific organic anion transporter 2 protein (CMOAT2) protein. The CMOAT2 protein is an inducible transporter in the biliary and intestinal excretion of organic anions and exports bile acids and glucuronides from cholestatic hepatocytes.

The microtubule associate serine/threonine kinase 3 (MAST3) (Gene ID: 23031) gene encodes the MAST3 protein. The MAST protein is serine/threonine kinase.

The TSPO associate protein (TSPOAP1) (Gene ID: 9256) gene encodes the peripheral-type benzodiazepine receptor-associated protein 1 (PTBRAP1, also known as RIMB-1) protein.

The CCM2 like scaffold protein (CCM2L) (Gene ID: 140706) gene encodes the CCM2L protein. The CCM2L protein prevents the activation of MEKK3 (MAP3K) kinase in vitro.

The mitogen-activated protein kinase kinase kinase 12 (MAP3K12) (Gene ID: 7786) gene encodes the MAP3K12 protein. The MAP3K12 protein is a member of the serine/threonine protein kinase family which is predominantly expressed in neuronal cells. The MAP3K12 protein forms heterodimers with leucine zipper-containing transcription factors, such as cAMP responsive element binding protein (CREB) and MYC, and thus may play a regulatory role in PKA or retinoic acid induced neuronal differentiation.

The protein kinase D2 (PRKD2) (Gene ID: 25865) gene encodes the PRKD2 protein. The PRKD2 protein is a serine/threonine protein kinase that converts transient diacylglycerol (DAG) signals into prolonged physiological effects downstream of PKC, and is involved in the regulation of cellular proliferation via MAPK1/3 (ERK1/2) signaling and oxidative stress-induced NF-kappa-B activation.

The selenoprotein W (SELENOW, also known as SEPW1) (Gene ID: 6415) gene encodes the selenoprotein W protein. The selenoprotein W protein is a glutathione-dependent antioxidant and may be involved in a redox-related process.

The SH3 and multiple Ankyrin repeat domain 3 (SHANK3) (Gene ID: 85358) gene encodes the SHANK3 protein. The SHANK3 protein encodes a major scaffold postsynaptic density protein which interacts with multiple proteins and complexes to orchestrate dendritic spine and synapse formation, maturation, and maintenance.

The collagen type XI alpha 2 chain (COL11A2) (Gene ID: 1302) gene encodes the COL11A2 protein. The COL11A2 protein encodes one of the two alpha chains of type XI collage, a minor fibrillar collagen.

The family with sequence similarity 81 member A (FAM81A) (Gene ID: 145773) gene encodes the FAM81A protein. The function of the FAM81A protein is unknown.

The family with sequence similarity 83 member H (FAM83H) (Gene ID: 286077) gene encodes the FAM83H protein. The FAM83H protein is critical in the structural development and calcification of tooth enamel.

The solute carrier family 46 member 1 (SLC46A1) (Gene ID: 113235) gene encodes the proton-coupled folate transporter (PCFT) protein. The PCFT protein is an intestinal proton-coupled high-affinity folate transporter and as an intestinal heme transporter which mediates heme uptake from the gut lumen into duodenal epithelial cells.

The multiple EGF like domains 8 (MEGF8) (Gene ID: 1954) gene encodes the MEGF8 protein. The MEGF8 protein is a single-pass type I membrane protein of unknown function.

The protein-O-mannosyltransferase 1 (POMT1) (Gene ID: 10585) gene encodes the POMT1 protein. The POMT1 protein is an O-mannosyltransferase that requires interaction with the POMT2 protein for enzymatic function. The POMT1 protein is found in the membrane of the endoplasmic reticulum.

The apoptosis associate tyrosine kinase (AATK) (Gene ID: 9625) gene encodes the AATK protein. The AATK protein is a tyrosine kinase that is induced during apoptosis and may be necessary for the induction of growth arrest and/or apoptosis of myeloid precursor cells.

The C-type lectin domain family 18 member A (CLEC18A) (Gene ID: 348174) gene encodes the CLEC18A protein. The CLEC18A protein binds polysaccharides in the presence of calcium and may regulate cell adhesion, the immune response, and apoptosis.

The myosin VIIB (MYO7B) (Gene ID: 4648) gene encodes the MYO7B protein. The MYO7B protein is found in brush border microvilli of epithelial cells in the intestines and kidneys. The MYO7B protein links protocadherins to the actin cytoskeleton and is essential for microvilli function.

The solute carrier family 5 member 10 (SLC5A10) (Gene ID: 125206) gene encodes the SLC5A10 protein. The SLC5A10 protein is a sodium/glucose transporter that has a high affinity for mannose and is expressed in the kidney.

The granzyme A (GZMA) (Gene ID: 3001) gene encodes the GZMA protein. The GZMA protein is a protease in the cytosolic granules of cytotoxic T cells and natural killer (NK) cells which activates caspase-independent cell death with morphological features of apoptosis when delivered into target cells through the immunological synapse.

The cystathionine gamma-lyase (CTH) (Gene ID: 1491) gene encodes the CTH protein. The CTH protein is a cytoplasmic enzyme in the trans-sulfuration pathway that converts cystathione derived from methionine into cysteine.

The collectrin, amino acid transport regulator (CLTRN, also known as TMEM27) (Gene ID: 57393) gene encodes the collectrin protein. Collectrin is a type I transmembrane protein that traffics amino acid transporters to the apical brush border of proximal tubules. Collectrin regulates insulin exocytosis by regulating formation of the soluble N-ethylmaleimide-sensitive-factor attachment protein receptor (SNARE) complex in pancreatic beta cells.

The carbonic anhydrase (CA7) (Gene ID: 766) gene encodes the CA7 protein. The CA7 protein is a cytosolic protein predominantly expressed in the brain and contributes to bicarbonate driven GABAergic neuron excitation.

The ABRA C-terminal like (ABRACL) (Gene ID: 58527) gene encodes the ABRACL protein. ABRACL is a protein of unknown function.

The prothymosin alpha (PTMA) (Gene ID: 5757) gene encodes the PTMA protein. The PTMA protein may mediate immune function by conferring resistance to certain opportunistic infections.

The pancreatic progenitor cell differentiation and proliferation factor (PPDPF) (Gene ID: 79144) gene encodes the PPDPF protein. The PPDPF protein may regulate exocrine pancreas development.

The mitochondrial calcium uniporter (MCU) (Gene ID: 90550) gene encodes the MCU protein. The MCU protein is a calcium transporter in the mitochondrial inner membrane that interacts with the mitochondrial calcium uptake 1 (MCU1) protein. The MCU protein constitutes the pore-forming and calcium-conducting subunit of the uniporter complex.

The homeodomain interacting protein kinase 3 (HIPK3) (Gene ID: 10114) gene encodes the HIPK3 protein. The HIPK3 protein is a serine/threonine protein kinase that regulates transcription, apoptosis, and steroidgenic gene expression. HIPK3 phosphorylates JUN and RUNX2 proteins.

The GTP cyclohydrolase I feedback regulator (GCHFR) (Gene ID: 2644) gene encodes the GCHFR protein. The GCHFR protein is a GTP cyclohydrolase I feedback regulatory protein that binds and regulates tetrahydrobiopterin inhibition of GTP cyclohydrolase I.

The cAMP responsive element modulator (CREM) (Gene ID: 1390) gene encodes the CREM protein. The CREM protein is a transcriptional regulator that binds the cAMP response element (CRE) sequence that is present in many viral and cellular promoters. CREM regulates spermatogenesis and spermatid maturation.

The protein tyrosine phosphatase type IVA, member 1 (PTP4A1) (Gene ID: 7803) gene encodes the PTP4A1 protein. The PTP4A1 is a tyrosine phosphatase that stimulates progression from G1 into S phase during mitosis. PTP4A1 may play a role in the development and maintenance of differentiating epithelial tissues and enhances cell proliferation, cell motility, and invasive activity.

The thiosulfate sulfur transferase (TST) (Gene ID: 7263) gene encodes the TST protein. The TST protein catalyzes the conversion of thiosulfate and cyanide to thiocyanate and sulfite in the mitochondria. TST also interacts with 5S ribosomal RNA and promotes its import into the mitochondria.

The immediate early response 3 (IER3) (Gene ID: 8870) gene encodes the radiation-inducible immediate-early gene IEX-1 (IEX1) protein. The IEX1 protein inhibits the dephosphorylation of ERK by phosphatase PP2A-PPP2R5C holoenzyme.

The GSKB interaction protein (GSKIP) (Gene ID: 51527) gene encodes the GSKIP protein. The GSKIP protein is a kinase-anchoring protein that binds and regulates the kinase activity of GSK3B and PKA. The ternary complex of GSKIP, GKS3B, and PKA enhances Wnt-induced signaling by facilitating the GSK3B- and PKA-induced phosphorylation of beta-catenin leading to beta-catenin degradation and stabilization respectively.

The chromosome 3 open reading frame 18 (C3ORF18) (Gene ID: 51161) encodes the C3OR18 protein. The function of the C3ORF18 protein is unknown.

The lysophospholipase I (LYPLA1) (Gene ID: 10434) gene encodes the LYPLA1 protein. The LYPLA1 protein hydrolyzes fatty acids from S-acylated cysteine residues in trimeric G alpha proteins.

The CUB and Sushi multiple domains 1 (CSMD1) (Gene ID: 64478) gene encodes the CSMD1 protein. The CSMD1 protein is a potential suppressor of squamous cell carcinomas.

The major histocompatibility complex (MHC) (Gene ID: 3107) gene encodes the MHC protein. The MHC protein is a heterodimer consisting of a heavy chain and a light chain. The heavy chain is anchored in the membrane. MHC play a central role in the immune system by presenting peptides derived from endoplasmic reticulum lumen.

The RAB38, member RAS oncogene family (RAB38) (Gene ID: 23682) gene encodes the RAB38 protein. The RAB38 protein regulates the peripheral melanosomal distribution of TYRP1 in melanocytes. RAB38 also regulates the maturation of phagosomes that engulf pathogens, melanin production, and melanosome biogenesis.

The cingulin like 1 (CGNL1) (Gene ID: 84952) gene encodes a member of the cingulin family. The CGNL1 protein localizes to both adherens and tight cell-cell junctions and mediates junction assembly and maintenance by regulating the activity of the small GTPases RhoA and Rac1.

The tudor domain containing 5 (TDRD5) (Gene ID: 163589) gene is required during spermiogenesis to participate in the repression transposable elements and prevent their mobilization, which is essential for the germline integrity. It likely acts via the piRNA metabolic process, which mediates the repression of transposable elements during meiosis by forming complexes composed of piRNAs and Piwi proteins and governs the methylation and subsequent repression of transposons.

### Pathway Genes

In some aspects, the present disclosure provides methods comprising contacting a kidney cell with an agent that modifies expression of or modifies activity of a product encoded by a pathway gene upstream from a gene selected from NDST3, CISH, SLCO1C1, SOCS2, SERPINC1, CGNL1, and TDRD5.

In some aspects, the present disclosure provides methods comprising contacting a kidney cell with an agent that modifies expression of or modifies activity of a product encoded by a pathway gene downstream from a gene selected from NDST3, CISH, SLCO1C1, SOCS2, SERPINC1, CGNL1, and TDRD5.

A pathway gene is an upstream gene or a downstream gene of a biological pathway in which a gene of interest functions. A pathway gene is considered upstream from a gene of interest when the pathway gene has an effect (direct or indirect) on the gene of interest. A pathway gene is considered downstream from a gene of interest when the gene of interest has an effect (direct or indirect) on the pathway gene.

The product of the NDST3 gene is part of the heparin sulfate biosynthesis pathway. In some embodiments, the pathway gene encodes a protein in the heparin sulfate biosynthesis pathway. Non-limiting examples of genes encoding proteins in the heparin sulfate biosynthesis pathway include B3GALT6, XYLT1, CYLT2, GLCE, EXTL3, EXTL2, DS3, NDST1, NDST2, and NDST4. Thus, in some embodiments, an agent of the present disclosure modifies expression of or modified activity of a product encoded by one or more genes selected from B3GALT6, XYLT1, CYLT2, GLCE, EXTL3, EXTL2, DS3, NDST1, NDST2, and NDST4.

The products of the CISH and SOCS2 genes suppress cytokine signaling. In some embodiments, the pathway gene encodes a protein in the cytokine signaling pathway. Non-limiting examples of cytokine genes include interleukin-2 (IL2), interferon (IFN), interleukin-10 (IL10), interleukin-1 (IL1), EPO, and THPO. Thus, in some embodiments, an agent of the present disclosure modifies expression of or modified activity of a product encoded by one or more genes selected from interleukin-2 (IL2), interferon (IFN), interleukin-10 (IL10), interleukin-1 (IL1), EPO, and THPO.

The SLCO1C1 gene is an organic anion transporter. In some embodiments, the pathway gene encodes a protein in the organic anion transporter pathway. Non-limiting examples of genes in the organic anion transporter pathway include SLCO4A1, SLCO1B3, SLCO3A1, SLC22A6, ABCC3, SLC22A8, and SLCO6A1. Thus, in some embodiments, an agent of the present disclosure modifies expression of or modified activity of a product encoded by one or more genes selected from SLCO4A1, SLCO1B3, SLC03A1, SLC22A6, ABCC3, SLC22A8, and SLCO6A1.

The product of the SERPINC1 gene is part of the blood coagulation pathway. In some embodiments, the pathway gene encodes a protein in the blood coagulation pathway. Non-limiting examples of genes encoding proteins in the blood coagulation pathway include PROC, F9, F5, 11, F12, F12A1, F8, F2, TFPI, and F2RL1. Thus, in some embodiments, an agent of the present disclosure modifies expression of or modified activity of a product encoded by one or more genes selected from PROC, F9, F5, F11, F12, F12A1, F8, F2, TFPI, and F2RL1.

The product of CGNL1 gene is part of the tight junction pathway. In some embodiments, the pathway gene encodes a protein in the tight junction pathway.

The product of TDRD5, at least in mice, binds piRNA precursors and selectively enhances pachytene piRNA processing (Ding D. et al. Nature Communications 2018; 9(127); and, it is required for retrotransposon silencing, chromatoid body assembly, and spermiogenesis (Yabuta Y. et al. JCB 2011; 192(5):781). Thus, a pathway gene herein may encode a protein involved in retrotransposon silencing, chromatoid body assembly, and/or spermiogenesis.

### EXAMPLES

### Example 1

### Sequencing and Assembly of the Black Bear Genome

The ability to explore the black bear genome for unique features and to facilitate gene expression analysis depends on an assembled and well-annotated genome. High-throughput sequencing of the black bear genome has been previously reported²³, but only using Illumina short-read sequencing to a ˜30× average depth of coverage. We improved this coverage by extracting DNA from a single male Maine black bear (JAX001) and sequencing it using three methods: paired-end sequencing and mate-pair sequencing using the Illumina HiSeq 2500 platform and single molecule sequencing using the PacBio RSII system (Table S2 and S3). Raw reads were assembled into 113,759 scaffolds and contigs ≥1000 bases with an N50 length of 190 Kb, totaling 2.59 Gb in length (data not shown). This is slightly larger than the estimated size of the panda bear assembly (2.46 Gb)²⁴ and the polar bear assembly (2.53 Gb)²³.

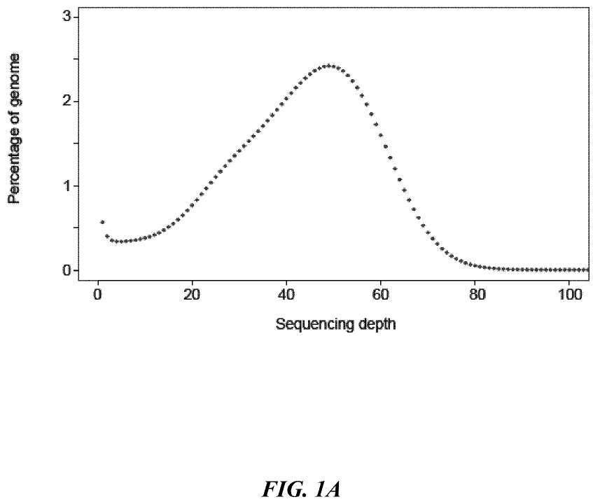

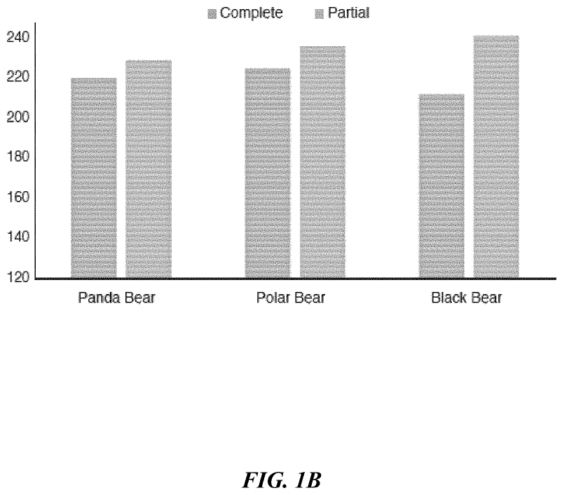

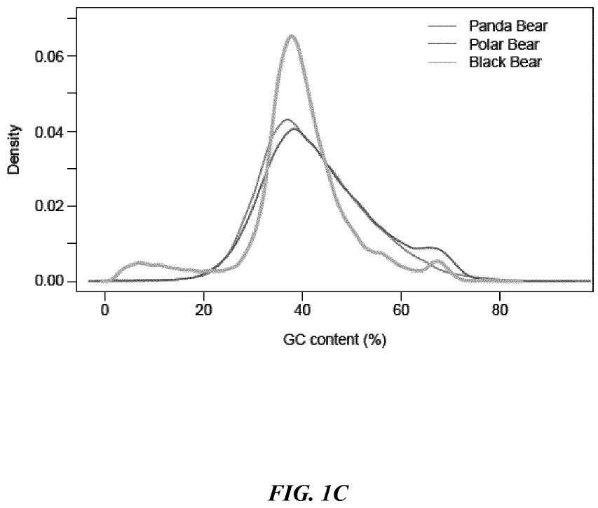

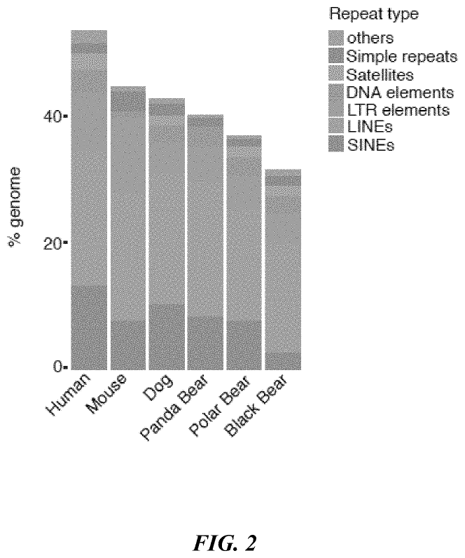

We estimated the quality of the assembled sequence by mapping all paired-end reads back to the assembled genome with Burrows-Wheeler Aligner²⁵ to determine the mapability and median coverage of the assembled genome. Approximately 90% of reads mapped back to the assembly with a mapping quality of ≥30 and with a median coverage of 43×. The peak sequencing depth was 50×, and more than 20 reads covered over 85% of the assembled sequences (FIG. 1A). The completeness of our assembly was estimated using CEGMA by screening against 248 highly conserved core eukaryotic genes. Our black bear assembly covers 212 out of 248 genes completely and 241 out of 248 genes partially. FIG. 1B shows this 85.5% completeness compared to 88.7% for the panda bear assembly and 90.7% for the polar bear assembly, which are all comparable. GC content in mammals is correlated with a number of genomic features that are functionally relevant, for example, gene density, transposable element distribution, and methylation rate. The GC-content distribution in the black bear covers a narrower range compared to the panda bear and the polar bear (FIG. 1C). This might be a consequence of the lower amount of repetitive sequences that is present in the black bear genome (FIG. 2). Thus, we were able to establish a genome assembly of good coverage and quality for the black bear, comparable to polar bear and panda bear.

### Example 2

### Annotation of the Black Bear Genome

Similar to the panda genome²⁶, our black bear genome assembly was annotated using the Comparative Annotation Toolkit (CAT). CAT identified 29,624 genes (18,091 coding) in our black bear assembly, representing 88% of the genes present in the dog annotation. In addition, 2,995 transcripts with at least 2 splices were predicted by AugustusCGP, which also had at least one intron junction supported by RNA-Seq and not supported by transMap. Of these, 1,730 were associated with a transMapped gene and assigned as a novel isoform of that gene. The remainder were evaluated for being putatively novel loci. Six hundred fifty three (653) loci were identified as being possibly paralogous, which is defined as overlapping a paralogous transMap projection, which was dropped in paralog resolution. Twenty eight (28) loci were identified as possible false fusions, defined as having >80% overlap with more than one transMap projection. Four hundred fifty eight (458) loci were identified as poor alignments, which are predictions in regions, which have alignment to a known gene but did not pass transMap filtering. This left 126 putatively novel loci. Of the 22,081 orthologous protein coding transcripts identified, pairwise codon-aware alignments of coding sequences identified frame-shifting indels in 7,848 transcripts. A slight enrichment of frame-shifting deletions was seen relative to insertions, suggesting a systematic bias in the assembly process.

### Example 3

### High-Throughput Sequencing of Renal RNA and Differential Expression Between Fall and Spring

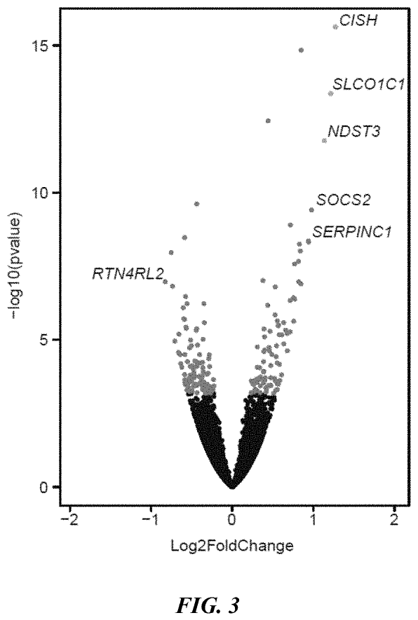

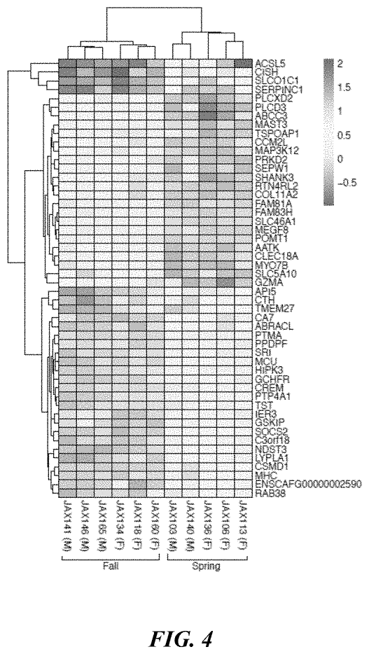

Kidneys were collected in the fall (animals 118, 134, 141, 146, 160, and 165), before hibernation, and in the spring (animals 101, 103, 106, 113, 136, and 140), shortly after the bears emerged from hibernation (3 males and 3 females for each time point). The timing of sample collection was (in part) driven by practicality. It was only possible to collect samples during a few specific periods, which were the late fall, before hibernation and early spring, approximately one or two weeks after the animals come out of hibernation. Approximately 60 million RNA-Seq reads were obtained from each sample before quality control (data not shown). After appropriate quality control and correction for batch effect, we performed a principal component analysis (data not shown) and determined that sample 101 (from a male in the spring) was an outlier (data not shown). After removing this sample and repeating the analysis (data not shown) we compared the spring samples to the fall samples. We identified 169 differentially expressed protein-coding genes with an adjusted P-value below 0.05, of which CISH, SLCO1C1, and NDST3 showed a >2-fold change (FIG. 3 and Table 1). Of these, 101 genes were downregulated and 68 genes were upregulated after hibernation. Fold changes ranged only from 1.8-fold downregulation (RTN4RL2) to 2.4-fold upregulation (CISH). FIG. 4 shows a heatmap for all 11 bears with the 50 most significant genes. Pathway enrichment analysis (Ingenuity Pathway Analysis) of the 169 genes did not identify any specific pathways. However, most notable was the upregulation of three cytokine suppression genes (SOCS2, CISH, and SERPINC1) and the lack of increased cytokine expression (e.g. IL6, CCL2, CCL6) and damage markers (LCN2 and HAVCR1) normally seen in lower functioning or recovering kidneys of other species²⁷.

TABLE 1
Summary of differentially expressed genes

| Gene Name | Fold change |
|---|---|
| RTN4RL2 | 0.563959823 |
| PLCD3 | 0.593562628 |
| ABCC3 | 0.600305343 |
| PLCXD2 | 0.612593477 |
| GZMA | 0.634248172 |
| SLC16A13 | 0.634289917 |
| C1QL1 | 0.644103232 |
| MELTF | 0.650384005 |
| SLC5A10 | 0.657724431 |
| MYO7B | 0.66114193 |
| SHANK3 | 0.66511619 |
| CBARP | 0.66573541 |
| CLEC18A | 0.665798772 |
| UNC5CL | 0.666707064 |
| SYT14 | 0.667268545 |
| SAMD11 | 0.667887127 |
| CATSPERB | 0.670461754 |
| gene_biotype=unknown_likely_coding | 0.670712332 |
| SEPW1 | 0.673744228 |
| AATK | 0.675428271 |
| CACNB4 | 0.678744586 |
| FBXO41 | 0.67877328 |
| PRKD2 | 0.680001311 |
| ATRNL1 | 0.682083198 |
| NECTIN1 | 0.68427657 |
| ENSCAFG00000028473 | 0.684356424 |
| PRELP | 0.688586575 |
| CXCL12 | 0.688701312 |
| NOXO1 | 0.689646655 |
| KCNJ12 | 0.692721338 |
| KIF26A | 0.693603772 |
| MAP3K12 | 0.695105125 |
| MLPH | 0.696323396 |
| GRIA3 | 0.697986356 |
| SLC22A6 | 0.698290862 |
| ENSCAFG00000032311 | 0.70041243 |
| CCM2L | 0.700438102 |
| COL11A2 | 0.705443772 |
| MMP11 | 0.706443795 |
| WASF3 | 0.709412525 |
| RGS11 | 0.709945119 |
| SLC22A23 | 0.71748087 |
| DIO1 | 0.717732394 |
| ASPG | 0.720615575 |
| SESN2 | 0.723154292 |
| GATA5 | 0.723228053 |
| PMM1 | 0.726091494 |
| NPR2 | 0.726152218 |
| VNN1 | 0.72885981 |
| PPIC | 0.733358127 |
| SLC46A1 | 0.735051978 |
| TCEA2 | 0.737766764 |
| FAM81A | 0.738328648 |
| MAST3 | 0.738490667 |
| SKI | 0.738873422 |
| ABCA1 | 0.740121939 |
| NAT9 | 0.741673056 |
| TSPOAP1 | 0.74345839 |
| WSCD1 | 0.756121619 |
| TMEM175 | 0.756813158 |
| NPTXR | 0.757155504 |
| TTC28 | 0.759838172 |
| C19orf44 | 0.760166434 |
| MYCBPAP | 0.76034286 |
| LNPK | 0.767308788 |
| PTPRJ | 0.770607124 |
| ANO8 | 0.773342942 |
| DOCK6 | 0.774300583 |
| AP5Z1 | 0.776129346 |
| HAUS3 | 0.776950088 |
| ACAA2 | 0.777398186 |
| FAM131C | 0.7781073 |
| POMT1 | 0.779494112 |
| GGT1 | 0.779552373 |
| FAM83H | 0.785707179 |
| KDM6B | 0.785724325 |
| CTTNBP2 | 0.786816788 |
| MEGF8 | 0.788115054 |
| COL18A1 | 0.794099113 |
| MZF1 | 0.794113068 |
| ALG1 | 0.796668424 |
| CDH2 | 0.797134704 |
| SMPD4 | 0.804966144 |
| CGNL1 | 0.807764567 |
| NCOR2 | 0.809644614 |
| ENSCAFG00000000579 | 0.812341338 |
| SMARCA1 | 0.813582767 |
| LMF1 | 0.816975895 |
| KMT2D | 0.818718628 |
| NEURL4 | 0.822531883 |
| MYH14 | 0.82277577 |
| CES2 | 0.822833377 |
| CERS2 | 0.823565868 |
| DENND4B | 0.824253594 |
| PEX6 | 0.824261599 |
| PLXNB2 | 0.831533417 |
| TMEM116 | 0.832505941 |
| DVL1 | 0.836431563 |
| SZT2 | 0.849573277 |
| GLG1 | 0.852073949 |
| EHMT2 | 0.856003469 |
| PSMD7 | 1.175264155 |
| NDUFB10 | 1.177084561 |
| SIPA1L1 | 1.194595348 |
| ELK4 | 1.199221287 |
| SNRPD3 | 1.203107132 |
| C2orf68 | 1.204978004 |
| GPKOW | 1.21410875 |
| UBE2K | 1.217774907 |
| FAM83B | 1.235443717 |
| EIF5 | 1.237613798 |
| CLIC5 | 1.237749582 |
| PPDPF | 1.23915647 |
| MOCS1 | 1.239513053 |
| HSPD1 | 1.264585978 |
| LIPT1 | 1.269080673 |
| MRPL20 | 1.269358823 |
| RILPL2 | 1.272468136 |
| KCTD10 | 1.272558618 |
| PTMA | 1.300357568 |
| GCDH | 1.30644209 |
| MORC4 | 1.30681867 |
| ACMSD | 1.306961426 |
| GNA13 | 1.310035213 |
| CFL2 | 1.310085541 |
| HIPK3 | 1.311487078 |
| TST | 1.317684884 |
| SRI | 1.318192082 |
| IVD | 1.325445269 |
| ASPH | 1.333167176 |
| SALL1 | 1.336324585 |
| DNAJC6 | 1.339042984 |
| IER3 | 1.354370787 |
| PTP4A1 | 1.358883143 |
| ENSCAFG00000029877 | 1.363115404 |
| CREM | 1.369816149 |
| TMEM27 | 1.380881395 |
| NCOA4 | 1.387207615 |
| RHOBTB1 | 1.389394001 |
| TRPS1 | 1.390071365 |
| EPHX2 | 1.390379745 |
| ALPL | 1.424996428 |
| NR1H4 | 1.434887159 |
| GCHFR | 1.442805973 |
| MCU | 1.444117489 |
| USP2 | 1.454972566 |
| gene_biotype=unknown_likely_coding | 1.459899463 |
| GLUD1 | 1.468403673 |
| KPNA7 | 1.469405126 |
| GSKIP | 1.470567611 |
| ENSCAFG00000002590 | 1.481737708 |
| TDRD5 | 1.483023154 |
| CTH | 1.484579035 |
| NUPR1 | 1.496991457 |
| NK4IN1 | 1.525004259 |
| C3orf18 | 1.551652305 |
| RAB38 | 1.553251418 |
| ABRACL | 1.578020888 |
| API5 | 1.640902549 |
| LYPLA1 | 1.643331277 |
| ACSL5 | 1.688534969 |
| CA7 | 1.703168767 |
| SERPINC1 | 1.704052482 |
| SOCS2 | 1.80132053 |
| CSMD1 | 1.969433697 |
| NDST3 | 2.19663451 |
| SLCO1C1 | 2.321343138 |
| CISH | 2.414780129 |

Because of the lack in inflammatory response, we asked whether the differentially expressed genes matched a particular development stage of the kidney. We used a method previously employed to place human cancers on a developmental landscape²⁸. First, we obtained gene expression profiles from mouse kidneys at different developmental stages (from E12.5 through E16.5) that were deposited in NCBI GEO and plotted the developmental expression profile in their first two temporal principal components (PC1-2). The high-dimensional expression profiles are simplified into a developmental timeline, ordering the genes in a linear array based on their temporal pattern of expression. Early genes are localized on the left end, genes with no bias towards early or late expression center in the middle and late genes localize the right end. Thus, the unique order of genes on the timeline represents a summary of early and late states for each developmental process. Next, we matched the differentially expressed bear genes with their mouse orthologs (153 out of 169 genes) and plotted their location on the developmental timeline for the genes that were expressed in the developing kidney (109 genes). We observed clustering of downregulated genes with the early mouse genes (E12.5) and clustering of upregulated genes with the late mouse genes (16.5) (data not shown). The processes that take place in the E12.5 kidney are focused on progenitor cell dynamics, fibroblast growth factor (FGF), and Wnt signaling, which we know are pro-fibrotic in the adult. In contrast, E16.5 is very different as it involves glomerulogenesis, tubulogenesis and maturation.

Taken together, despite the many months of reduced renal function and down-regulated metabolism during hibernation, the gene expression differences between spring and fall bears is limited both in number of genes that are differentially expressed, fold-change, and the lack of upregulation of genes that are typically seen in other species after a period of low function or damage. In fact, the expression profile of bears coming out of hibernation resembles a developmental stage.

### Example 4

### Identification of RNA-Editing in the Black Bear Kidney

Our RNA-Seq data identified possible RNA-editing in a small set of genes when aligning the reads to our assembled genome sequence. In order to rule out the possibility of variation in the transcripts due to single nucleotide polymorphisms (SNPs) in the genomes of the bears for which we performed the RNA-Seq, we sequenced the genomes of bears 101, 103, 113, 118, 134, and 160 at a low coverage (data not shown). After comparing with the genome sequences and filtering for protein-coding transcripts we identified 38 transcripts containing a different variant (between 10% and 95% of the total reads) from the genome sequence (Table 2). Of these 38 transcripts, 30 (79%) show A-to-I editing, while the other sites have C-to-U editing. Almost half of the edited sites are in the coding region and five are predicted to lead to an amino acid change.

According to the REDIportal (srv00.recas.ba.infn.it/atlas/) RNA-editing has been observed in four of the genes in the human kidney (although not at the same base pair) and for one gene, FLNB, RNA-editing also occurs both in the human and the mouse kidney. Interestingly, RNA-editing in 37 of the transcripts is observed in most or all 12 bears, but for ZNF688 we find editing in all 6 spring bears and no editing in the 6 fall bears.

To confirm our observations in the high-throughput sequencing data, we randomly selected eight editing sites and designed primers flanking these sites that could discriminate between RNA and possible genomic DNA contamination. Subsequent Sanger sequencing confirmed all eight editing sites (Table 2).

TABLE 2
Summary of coding transcripts with RNA-editing

| Gene | Contig | Position | Ref | Alt | Feature | AA change | Remark* |
|---|---|---|---|---|---|---|---|
| PTCD1 | 3060 | 216894 | A | I | 5′UTR | | |
| SYNE2 | 11596 | 1608 | A | I | CDS | L-to-L | |
| IDH1 | 660 | 132419 | C | U | 3′UTR | | |
| NMNAT1 | 7555 | 90746 | A | I | 3′UTR | | |
| SLC25A51 | 408 | 343378 | A | I | 5′UTR | | |
| KCNIP2 | 283 | 43560 | A | I | 3′UTR | | |
| PAICS | 3388 | 17253 | A | I | 3′UTR | | Human |
| TOR1AIP2 | 8765 | 12215 | A | I | 3′UTR | | |
| TRPM3 | 3807 | 132928 | C | U | 3′UTR | | |
| ENSCAFG00000040594 | 20747 | 50545 | A | I | exon | | |
| FLNB | 1255 | 165746 | C | U | CDS | M-to-V | Human, Mouse |
| ALDH1A1 | 26346 | 24965 | A | I | CDS | G-to-G | |
| HMGA1 | 10323 | 68028 | A | I | 3′UTR | | |
| ENSCAFG00000013809 | 267 | 1095 | A | I | CDS | | |
| IST1 | 818 | 78551 | A | I | 3′UTR | | |
| ZHX2 | 4067 | 112564 | A | I | CDS | K-to-R | |
| GPRC5B | 4938 | 128686 | A | I | CDS | T-to-T | Human |
| MICAL3 | 8064 | 249651 | A | I | 3′UTR | | |
| ENSCAFG00000022297 | 1496 | 321379 | A | I | exon | | |
| CPN2 | 20 | 146998 | A | I | CDS | O-to-R | |
| NBEAL2 | 179 | 269038 | A | I | CDS | A-to-A | |
| HOMER2 | 432 | 515746 | C | U | 3′UTR | | |
| ENSCAFG00000014154 | 440 | 66585 | A | I | exon | | |
| PLEKHM3 | 660 | 328758 | C | U | CDS | D-to-D | |
| USP39 | 875 | 493475 | C | U | CDS | Y-to-Y | |
| NFKB1 | 1339 | 289908 | A | I | CDS | A-to-A | |
| USP46 | 1582 | 132480 | A | I | 3′UTR | | |
| TMED3 | 2155 | 45921 | A | I | 3′UTR | | |
| FEM1B | 3491 | 152656 | A | I | 5′UTR | | |
| ENSCAFG00000030572 | 4212 | 309670 | A | I | exon | | |
| WWTR1 | 5646 | 16741 | A | I | 3′UTR | | Human |
| ZNF217 | 5967 | 51298 | A | I | CDS | K-to-R | |
| CEP95 | 7197 | 19854 | A | I | 3′UTR | | |
| CTSO | 7856 | 89886 | A | I | CDS | T-to-A | |
| MPP7 | 9440 | 111224 | C | U | 3′UTR | | |
| GTPBP1 | 10248 | 93709 | A | I | CDS | T-to-T | Human |
| ZNF688 | 76864 | 76864 | A | I | 3′UTR | | |

Previous studies in bears suggest they have unique features in the kidney that allow them to endure lower renal functioning during hibernation and recovery soon after hibernation. As these features are likely in part encoded in the genome sequence and gene expression patterns unique to the bear, we set out to explore the genome sequence and renal gene expression of the American black bear (Ursus americanus).

A first step was to establish the complete annotated genome sequence. In order to accomplish this, we used three approaches for sequencing a single male bear with 100× coverage before filtering. This resulted in a final, high quality, assembly. This current genome is comparable in quality and size to the published panda genome²⁴ and polar bear genome.²³ A comparison between the three genomes shows a narrower range in GC-content in the black bear (FIG. 1C), likely the result of fewer repetitive sequences caused by transposon integration (FIG. 2).

The new Comparative Annotation Toolkit (CAT) pipeline allowed us to establish a high-quality annotation of the genome with 22,081 protein coding transcripts and identification of 126 putative novel loci that warrant further investigation as to their uniqueness and possible function.

Based on the reported changes in the bear kidney function during hibernation,⁵ we expected we would find a signature of these changes in the gene expression of kidneys collected soon after hibernation when compared to kidneys collected before hibernation. We therefore collected samples from 3 male and 3 female bears (samples were collected within 10 days from each other) soon after hibernation and 3 male and 3 female bears before hibernation. One limitation is that the time between waking up from hibernation and sample collection is unknown and we assumed it was similar for all animals. Deviation from this assumption is expected to lead to more variation in the gene expression profile. RNA-Seq analysis identified 169 protein coding genes that were differentially expressed between the two time points. Of these, 101 genes were downregulated and 68 genes were upregulated after hibernation. Fold changes only ranged from 1.8-fold downregulation (RTN4RL2) to 2.4-fold upregulation (CISH) and only three genes showed a >2-fold change (CISH, SLCO1C1, and NDST3). Little is known about the function in the kidney of these genes: CISH is a member of the SOCS family and involved in suppression of cytokine signaling through the JAK-STAT5 pathway. SLCO1C1 is a member of the organic anion transport family and best known for mediating sodium-independent uptake of thyroid hormones in brain tissue. NDST3 is a member of the heparan sulfate/heparin GlcNAc N-deacetylase/N-sulfotransferase family and increased expression indicates an increase of N-sulfation of heparin sulfate in the kidney. Since kidney glucosaminoglycans have been shown to be related to the ability of turtles to conserve water,²⁹ it can be speculated that the increased expression of NDST3 in hibernating black bear kidneys protect the animals during the long period of dehydration during winter sleep. Moreover, since the endothelial glycocalyx on the vascular endothelial cells plays a major role in albuminuria and kidney disease,³⁰ it could be speculated that upregulation of NDST3 may protect the endothelial glycocalyx in bear kidneys during the anuric hibernation.

Most notable among the differentially expressed genes was the upregulation of cytokine suppression genes (SOCS2, CISH, and SERPINC1) in the post-hibernating bears and the lack of increased cytokine expression (such as IL6 and CCL2) and damage markers such as HAVCR1 (KIM-1) and LCAN (NGAL). Pathway enrichment analysis (IPA) did not identify any pathways that were significantly overrepresented among the differentially expressed genes. These findings support previous observation that despite several pro-inflammatory risk factors during the hibernation period (such as kidney dysfunction and immobilization), there are no signs of systemic inflammation³¹. This is further supported by our principal component analysis in which the genes that are downregulated after hibernation cluster with a developmental stage in which FGF and Wnt signaling, which we know are pro-fibrotic in the adult kidney, play an important role and genes that are upregulated after hibernation cluster with a developmental stage in which glomerulogenesis, tubulogenesis and maturation are important. In patients with CKD, systemic inflammation is a common finding that promotes premature aging and predicts poor outcome³².

Although it has been shown that American black bears maintain a reduced metabolic rate for up to three weeks after emerging from their dens³, a possible explanation for our results is that the kidneys already completely recovered from hibernation within the short time between emerging from hibernation and sample collection. Another explanation is that the reduction in metabolic rate and urine production has a much smaller impact on the kidney than would be predicted.

Our RNA-Seq data also identified the presence of RNA editing, which was confirmed for all sites that were tested with Sanger sequencing. Several of the genes that are edited also show RNA-editing in mouse and human, but at different positions (Supplemental Table 8). The most interesting is the RNA editing in the 3′UTR of the transcriptional regulator ZNF688, which only seems to happen in the spring. This suggests season-dependent RNA-editing and a possible mechanism through which gene expression is altered.

In conclusion, we established a high-quality and well-annotated genome sequence of the black bear (Ursus americanus). RNA-Seq of kidney samples before and after hibernation shows RNA-editing and the modest differential expression of a set of 169 genes that might be involved in the unique stress response due to hibernation in the bear. Our results suggest that during hibernation, changes in gene expression favors anti-inflammatory pathways.

### Materials and Methods

### Sample Collection, Library Preparation and Sequencing

Bear samples were obtained by hunters during the hunting seasons in Maine. Hunters were asked to participate on a voluntary base and no bears were killed for the specific purpose of this study. All methods were carried out in accordance with relevant guidelines and regulations. DNA was isolated using the DNeasy™ Blood & Tissue Kit (Qiagen®). The whole-genome library was prepared using the KAPA Hyper Prep Kit (Kapa Biosystems, Inc., Wilmington, Mass.) with a bead-based size selection to select for inserts with an average size of 400 bp and 6 cycles of PCR. Sequencing was done on two 2×125 bp Illumina 2500 lanes. The mate pair library was prepared using the Illumina Nexera Mate Pair Kit (Illumina, San Diego, Calif., USA) with a gel-based size selection to select for inserts with an average size of 10 kb and 14 cycles of PCR. Sequencing was done on two 2×100 bp Illumina 2500 lanes. The PacBio library was prepared using the Pacific Biosciences SMRTbell Template Prep Kit 1.0 (Pacific Biosciences, Menlo Park, Calif., USA) using the “20-kb Template Preparation Using BluePippin Size-Selection System (15-kb Size cutoff)” protocol obtained from PacBio SampleNet. The BluePippin was set to collect from 7-50 kb. Sequencing was done on 23 PacBio SMRT cells. For RNA-Seq, hunters collected kidneys and stored them in liquid nitrogen. Kidneys were first homogenized in Trizol (Invitrogen) and an aliquot was used for RNA isolation using the miRNeasy Mini kit (Qiagen), according to manufacturers' protocols, including the optional DNase digest step. Quality was assessed using an Agilent 2100 Bioanalyzer instrument and RNA 6000 Nano LabChip assay (Agilent). We prepared total RNA for sequencing using the Illumina TruSeq methodology (TruSeq Stranded Total RNA LT Sample Prep Kit with Ribo-Zero Gold). The first step involves the removal of ribosomal RNA (rRNA) using biotinylated, target-specific oligos, combined with Ribo-Zero rRNA removal beads. After individual samples were bar-coded they were pooled and sequencing was done on two 2×100 bp Illumina 2500 lanes. All raw data has been submitted to the NCBI's Sequence Read Archive (ncbi.nlm.nih.gov/sra) under accession number SRP075217.

### Sequence Assembly

All data was subjected to quality control check⁹ and samples with base qualities greater ≥30 over 70% of read length were used in the downstream analysis. KmerFreq_HA v2.0 and Corrector_HA v2.01 tools of SOAPec_v2.01¹⁰ were further used to perform K-mer frequency generation and error correction of paired-end and mate-pair data, respectively. SOAPdenovo-127mer (v2.04)¹⁰ was used to perform the contig and scaffold assembly from paired end and mate-pair libraries (avg_ins=350 and 10K for paired-end and mate-pair, respectively). After the assembly, gaps in scaffolds were closed with the GapCloser tool (v1.12)¹⁰ (with—1 125) of the soap module. Smaltmap, perfect from bam and pipeline tools of the reapr module (v 1.0.17)¹¹ was used to recognize the errors in the assembly by re-mapping of paired-end and mate-pair data to the de novo assembled genome. The assembly was broken at potential misassembled points. The broken assembly was further used as an input for the jelly tool of the PBSuite¹² (v15.2.20) with blasr (v1.3.1) at parameters [-minMatch 8-minPctIdentity 80-bestn 1-nCandidates 20-maxScore-500]. This tool was used to upgrade the existing Illumina assembly with low-pass PacBio data. Repeat masking was performed by downloading the Repbase TE library from the repbase server (girinst.org/server/RepBase/). To identify known TE elements, we used repeat masker and repeat protein mask software in the Repeat Masker package (repeatmasker.org), which identifies TEs by aligning the genome sequence to a defined TE database. Tandem repeats were predicted using TRF¹³ by using the default parameters “Match=2, Mismatch=7, Delta=7, PM=80, PI=10, Minscore=50 and MaxPeriod=12. The completeness of the assembly was estimated by using CEGMA¹⁴. The assembly was screened against a collection of 248 universal eukaryotic single-copy genes. Core eukaryotic gene datasets were downloaded and a blast database was made from the assembly before running CEGMA. This Whole Genome Shotgun project has been deposited at DDBJ/ENA/GenBank under the accession LZNR00000000. Version LZNR01000000 is described in this work.

### Annotation

The black bear genome assembly was annotated using Comparative Annotation Toolkit (CAT—github.com/ComparativeGenomicsToolkit/Comparative-Annotation-Toolkit). CAT uses whole genome alignments generated by progressiveCactus¹⁵ to project gene annotations from a high quality reference genome on to one or more target genomes. This process leverages previously curated annotation sets to rapidly construct a set of orthologs in the target genomes. After transcript projection¹⁶, the protein coding projections are provided to the ab-initio gene finding tool AUGUSTUS¹⁷ with additional extrinsic information derived from RNA-seq. AUGUSTUS then enforces a coding gene model that allows for shifts in splice sites in order to maintain frame. This process can also rescue exons that did not align in the whole genome alignment. CAT also performs true ab-initio gene prediction by using a new method of running AUGUSTUS called Comparative Augustus, or AugustusCGP¹⁸. This parameterization simultaneously predicts genes in all species in a progressiveCactus alignment, using RNA-seq data in one or more species to help guide the annotation process. After projection, clean-up and ab-initio prediction, CAT combines these separate transcript sets into one final gene set through a consensus finding process. For orthologous transcripts, the transcript with the best fidelity to the reference with the best extrinsic support is chosen. In the case where multiple paralogous projections are found, CAT selects the most likely ortholog through a combination of splice junction fidelity, synteny and alignment identity. Finally, ab-initio predictions from AugustusCGP are evaluated for providing new information. If significant overlap with a orthologous projection is found, and the transcript provides a new splice junction or exon, then it is included as a new isoform of the ortholog. Otherwise, the locus is considered a candidate for a novel gene, often a gene family expansion. In addition to an annotation set on target genomes, CAT produces a UCSC Assembly Hub¹⁹. This assembly hub has tracks for the raw transMap projections, the post-filtering projections, the various modes of AUGUSTUS employed, the final consensus annotation set, as well as the input RNA-seq information including a filtered splice junction track. All annotations are stored in a modified bigBed format with a wide variety of various additional features annotated on each entry, which can be accessed from the details page for that entry. Additional features include RNA-seq support for specific splice junctions and binary classifiers such as having a frame-shifting indel relative to the source transcript. In addition to the genome-specific tracks, an alignment track (snake track) shows the cactus alignment between the current species and other species. Browsing this assembly hub provides the opportunity to visualize the relationship between all aligned species and the various ways the transcript projections were filtered and combined with ab-initio predictions. Assembly hub can be visualized by visiting UCSC “Track Data Hubs”

(genome.ucsc.edu/index.html) and then adding URL ftp://ftp.jax.org/maine_blackbear_project/assemblyHub/hub.txt under the “My Hubs” tab. For the black bear project, the cactus alignment generated contained black bear, horse (equCab2), dog (canFam3), polar bear (GCA_000687225.1), elephant (loxAfr3), human (hg38) and mouse (mm10) using the following guide phylogenetic tree: ((Human:0.145908,Mouse: 0.356483) human_mouse_anc:0.020593, (Horse:0.109397, (Dog:0.052458, (Polar_bear:0.01, Maine_black_bear:0.01) bear_anc:0.08) dog_bear_anc:0.069845) horse_dog_bear_anc:0.043625) root; CAT was run using the Dog Ensembl V87 annotation set as the reference. RNA-seq was obtained from SRA for dog (SRR2960309, SRR2960315, SRR2960317, SRR2960319, SRR2960320, SRR2960321, SRR2960328, SRR3727716, SRR3727717, SRR3727718, SRR3727719, SRR3727720, SRR3727722, SRR3727723, SRR3727724, SRR3727725, SRR5018836) and for polar bear (SRR950076, SRR950074) and aligned to their respective genomes. Additionally, RNA-seq was generated for Maine black bear as part of this project.

### Gene Expression Analysis and RNA Editing

Samples were aligned to the soft masked Bear Assembly using STAR aligner (v2.5.3) (—sjdbOverhang 75—quantMode GeneCounts—twopassMode Basic) with known annotation. STAR provided the expression count matrix was used for differential expression analysis with DESeq2. The genes with FDR<=0.05 were considered as differentially expressed between spring and fall Samples. STAR generated bam were further processed by picard MarkDuplicates and GATK SplitNCigarReads to remove the duplicates and split reads into exonic segments and hard clip any reads hanging into the intronic region, respectively. The final bam of all twelve samples were used to perform the join variant calling by HaplotypeCaller (v3.4-0). The output of HaplotypeCaller was further processed to identify the sites showed alternate allele in all twelve samples or that were genotype as reference in all six samples of one season and as alternate allele in the other season. We further filtered variants to set of canonical editing sites (and their reverse complementary). RNA editing prediction tool-REDItools was used to confirm the filtered sites, strand identification and its count was used as final count to estimate the editing frequency. These sites were subjected to confirmation in DNA-seq data generated for six samples. The whole genome sequence data of these bears were subjected to quality trimming and aligned by bwa-mem (v0.7.9a). The alignments were converted to bam and processed by Picard-MarkDuplicates to remove the duplicates from the data. Afterwards, the Genome Analysis tool kit module IndelRealigner was used to pre-process the alignments. The realigned bam file was processed to identify the coverage at potential editing sites. The sites with at least one DNA-seq sample support were included in final set for experimental validation. Gene expression profiles from embryonic mouse kidneys were downloaded from NCBI GEO

(ncbi.nlm.nih.gov/geo/query/acc.cgi?acc=GSE3808)²⁰. Six samples representing three time points (two replicates each) were used in the analysis. For the analysis, E12.5, E13.5 and E16.5 were considered early & late age samples, respectively. GEO samples accessions of array data are GSM87387, GSM87388, GSM87389, GSM87390, GSM87391, and GSM87392. Samples were normalized using the affy package²¹ and GenBank accession for the probes (Affymetrix® Mouse Expression 430A Array) were downloaded from GEO (www.ncbi.nlm.nih.gov/geo/query/acc.cgi?acc=GPL339). We extracted the MGI Gene IDs corresponding to GenBank accession using mouse genome informatics database (http://www.informatics.jax.org), performed the principal component analysis (PCA) on the mouse array data and created a PCA biplot (Supplemental Table S10). To map the differentially expressed (DE) bear genes on the PCA biplot, we first extracted the corresponding dog gene Ensembl id from the bear generic feature format file (gff) and extracted the Ensembl id of mouse gene orthologs of dog from biomart²². Only genes with homology type: “ortholog_one2one”, orthology confidence:high and Query:target & target:query identity (≥50 percent) between dog and mouse were selected (153 genes remaining at the end) for mapping. Finally, we extracted the MGI:ID for the mouse ensembl id and mapped it on the mouse PCA biplot to create an eggogram (data not shown) and performed an analysis for significance (data not shown).

### Example 5

### Comparison of Genes Differentially Expressed Before and After Hibernation in Black Bears and in Tenrecs

The RNA from the kidneys of six tenrecs (3 collected before hibernation and 3 after hibernation) were used to create RNAseq libraries, e.g., as described above for the black bear studies. The libraries were sequenced paired end (2×76 bp) on an Illumina NextSeq. Sequenced reads (averaging 84 million reads per sample) were aligned to the Echinops telfairi (lesser hedgehog tenrec) genome (Ensembl TENREC, July 2005 assembly). Expression levels were compared before and after hibernation and may be involved in regeneration of the kidney post hibernation.

The tenrec dataset was then compared with the black bear dataset described above for genes that were present in both sets and showed an expression change in the same direction. Two genes meeting these criteria CGNL1 and TDRD5. CGNL1 encodes the Cingulin Like 1 protein, which is expressed at lower levels after hibernation, and TDRD5 encodes the Tudor Domain Containing 5 protein, which is expressed at higher levels after hibernation.

### REFERENCES

1. Levin, A., Tonelli, M., Bonventre, J., et al. 2017, Global kidney health 2017 and beyond: a roadmap for closing gaps in care, research, and policy. Lancet, 390, 1888-917.
2. Stenvinkel, P., and Johnson, R. J. 2013, Kidney biomimicry—a rediscovered scientific field that could provide hope to patients with kidney disease. Arch. Med. Res., 44, 584-90.
3. Tøien, Ø., Blake, J., Edgar, D. M., Grahn, D. A., Heller, H. C., and Barnes, B. M. 2011, Hibernation in black bears: independence of metabolic suppression from body temperature. Science, 331, 906-9.
4. Hellgren, E. C. 1998, Physiology of hibernation in bears. Ursus, 10, 467-77.
5. Nelson, R. A., Wahner, H. W., Jones, J. D., Ellefson, R. D., and Zollman, P. E. 1973, Metabolism of bears before, during, and after winter sleep. Am J Physiol, 224, 491-6.
6. Barboza, P. S., Farley, S. D., and Robbins, C. T. 2011, Whole-body urea cycling and protein turnover during hyperphagia and dormancy in growing bears (Ursus americanus and U. arctos). Canadian Journal of Zoology, 75, 2129-36.
7. Brown, D. C., Mulhausen, R. O., Andrew, D. J., and Seal, U. S. 1971, Renal function in anesthetized dormant and active bears. Am J Physiol, 220, 293-8.
8. Seger, R. L., Cross, R. A., Rosen, C. J., et al. 2011, Investigating the mechanism for maintaining eucalcemia despite immobility and anuria in the hibernating American black bear (Ursus americanus). Bone, 49, 1205-12.
9. Patel, R. K., and Jain, M. 2012, NGS QC Toolkit: a toolkit for quality control of next generation sequencing data. PLoS ONE, 7, e30619.
10. Luo, R., Liu, B., Xie, Y., et al. 2012, SOAPdenovo2: an empirically improved memory-efficient short-read de novo assembler. Gigascience, 1, 18.
11. Hunt, M., Kikuchi, T., Sanders, M., Newbold, C., Berriman, M., and Otto, T. D. 2013, REAPR: a universal tool for genome assembly evaluation. Genome Biol, 14, R47.
12. English, A. C., Richards, S., Han, Y., et al. 2012, Mind the gap: upgrading genomes with Pacific Biosciences RS long-read sequencing technology. PLoS ONE, 7, e47768.
13. Benson, G. 1999, Tandem repeats finder: a program to analyze DNA sequences. Nucleic Acids Research, 27, 573-80.
14. Parra, G., Bradnam, K., and Korf, I. 2007, CEGMA: a pipeline to accurately annotate core genes in eukaryotic genomes. Bioinformatics, 23, 1061-7.
15. Paten, B., Earl, D., Nguyen, N., Diekhans, M., Zerbino, D., and Haussler, D. 2011, Cactus: Algorithms for genome multiple sequence alignment. Genome Res, 21, 1512-28.
16. Stanke, M., Diekhans, M., Baertsch, R., and Haussler, D. 2008, Using native and syntenically mapped cDNA alignments to improve de novo gene finding. Bioinformatics, 24, 637-44.
17. Stanke, M., Schöffmann, O., Morgenstern, B., and Waack, S. 2006, Gene prediction in eukaryotes with a generalized hidden Markov model that uses hints from external sources. BMC Bioinformatics, 7, 62.
18. König, S., Romoth, L. W., Gerischer, L., and Stanke, M. 2016, Simultaneous gene finding in multiple genomes. Bioinformatics, 32, 3388-95.
19. Nguyen, N., Hickey, G., Raney, B. J., et al. 2014, Comparative assembly hubs: web-accessible browsers for comparative genomics. Bioinformatics, 30, 3293-301.
20. Schwab, K., Hartman, H. A., Liang, H.-C., Aronow, B. J., Patterson, L. T., and Potter, S. S. 2006, Comprehensive microarray analysis of Hoxa11/Hoxd11 mutant kidney development. Dev Biol, 293, 540-54.
21. Gautier, L., Cope, L., Bolstad, B. M., and Irizarry, R. A. 2004, affy—analysis of Affymetrix GeneChip data at the probe level. Bioinformatics, 20, 307-15.
22. Kinsella, R. J., Kähäri, A., Haider, S., et al. 2011, Ensembl BioMarts: a hub for data retrieval across taxonomic space. Database (Oxford), 2011, bar030.
23. Miller, W., Schuster, S. C., Welch, A. J., et al. 2012, Polar and brown bear genomes reveal ancient admixture and demographic footprints of past climate change. Proceedings of the National Academy of Sciences, 109, E2382-90.
24. Li, R., Fan, W., Tian, G., et al. 2010, The sequence and de novo assembly of the giant panda genome. Nature, 463, 311-7.
25. Li, H., and Durbin, R. 2009, Fast and accurate short read alignment with Burrows-Wheeler transform. Bioinformatics, 25, 1754-60.
26. Chen, M., Hu, Y., Liu, J., et al. 2015, Improvement of genome assembly completeness and identification of novel full-length protein-coding genes by RNA-seq in the giant panda genome. Sci Rep, 5, 18019.
27. Kumar, S., Liu, J., and McMahon, A. P. 2014, Defining the acute kidney injury and repair transcriptome. Semin Nephrol, 34, 404-17.
28. Kho, A. T., Zhao, Q., Cai, Z., et al. 2004, Conserved mechanisms across development and tumorigenesis revealed by a mouse development perspective of human cancers. Genes & Development, 18, 629-40.
29. Kupchella, C. E., Heckman, J. R., Lega, R. D., and Plamp, C. E. 1977, The influence of cold-torpor and water deprivation on plasma and kidney glycosaminoglycans in emydine turtles. Comp Biochem Physiol A Comp Physiol, 56, 225-9.
30. Liew, H., Roberts, M. A., MacGinley, R., and McMahon, L. P. 2017, Endothelial glycocalyx in health and kidney disease: Rising star or false Dawn? Nephrology (Carlton), 22, 940-6.
31. Stenvinkel, P., Fröbert, O., Anderstam, B., et al. 2013, Metabolic changes in summer active and anuric hibernating free-ranging brown bears (Ursus arctos). PLoS ONE, 8, e72934.
32. Kooman, J. P., Dekker, M. J., Usvyat, L. A., et al. 2017, Inflammation and premature aging in advanced chronic kidney disease. AJP: Renal Physiology, 313, F938-50.
33. Fedorov, V. B., Goropashnaya, A. V., Tøien, Ø., et al. 2011, Modulation of gene expression in heart and liver of hibernating black bears (Ursus americanus). BMC Genomics, 12, 171.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

1. A method of identifying an agent that modifies expression of a renal disease target, the method comprising:
contacting a kidney cell with an agent of interest; and
assaying the kidney cell for expression of a gene and/or activity of a product encoded by a gene listed in Table 1.

2. The method of claim 1, wherein the method comprises assaying for a change in expression of the gene and/or activity of a product encoded by a gene, relative to a control or relative to baseline.

3. The method of claim 2, wherein the change is an increased level of expression of the gene and/or activity of a product encoded by the gene.

4. The method of claim 3, wherein the change is a decreased level of expression of the gene and/or activity of a product encoded by the gene.

5. The method of claim 1, further comprising identifying the agent of interest as a candidate therapeutic agent for treating a renal disease if the change in expression and/or activity is at least 1.5-fold, relative to a control or relative to baseline.

6. The method of claim 5, further comprising identifying the agent of interest as a candidate therapeutic agent for treating a renal disease if the change in expression and/or activity is 1.5-fold to 3-fold, relative to a control or relative to baseline

7. The method of claim 1, wherein the control is expression of the gene and/or activity of a product encoded by the gene assayed following contacting a similar kidney cell with an inert substance.

8. A method of treating a subject having a renal disease, comprising:
delivering to the subject an agent that modifies expression of a gene or activity of a product encoded by a gene listed in Table 1.

9. The method of claim 1, wherein the agent modifies (a) expression of a gene selected from ACSL5, CISH, SLCO1C1, SERPINC1, PLCXD2, PLCD3, ABCC3, MAST3, TSPOAP1, CCM2L, MAP3K12, PRKD2, SEPW1, SHANK3, RTN4RL2, COL11A2, FAM81A, FAM83H, SLC46A1, MEGF8, POMT1, AATK, CLEC18A, MYO7B, SLC5A10, GZMA, API5, CTH, TMEM27, CA7, ABRACL, PTMA, PPDPF, SRI, MCU, HIPK3, GCHFR, CREM, PTP4A1, TST, IER3, GSKIP, SOCS2, C3ORF18, NDST3, LYPLA1, CSMD1, MHC, ENSCAFG00000002590, CGNL1, TDRD5, RAB38 and homologs thereof.

10. The method of claim 9, wherein the agent modifies (a) expression of a gene selected from NDST3, CISH, SLCO1C1, SOCS2, SERPINC1, CGNL1, TDRD5, and RTN4RL2, or (b) activity of a product encoded by a gene selected from NDST3, CISH, SLCO1C1, SOCS2, and SERPINC1, CGNL1, TDRD5, and RTN4RL2.

11. The method of claim 10, wherein the agent increases (a) expression of a gene selected from NDST3, CISH, SLCO1C1, SOCS2, TDRD5, and SERPINC1, or (b) activity of a product encoded by a gene selected from NDST3, CISH, SLCO1C1, SOCS2, TDRD5, and SERPINC1.

12.-17. (canceled)

18. The method of claim 11, wherein the expression or the activity is increased by at least 1.5 fold.

19. (canceled)

20. The method of claim 10, wherein the agent decreases (a) expression of RTN4RL2 or CGNL1, or (b) activity of a product encoded by RTN4RL2 or CGNL1.

21.-22. (canceled)

23. The method of claim 20, wherein the expression or the activity is decreased by at least 1.5 fold.

24. The method of claim 1, wherein the kidney cell is a black bear kidney, a mouse kidney cell, a tenrec kidney cell, or a human kidney cell.

25. The method of claim 24, wherein the kidney cell is a human kidney cell.

26.-27. (canceled)

28. The method of claim 1, wherein the gene is selected from NDST3, CISH, SLCO1C1, SOCS2, SERPINC1, CGNL1, and TDRD5.

29. The method of claim 8, wherein the gene is selected from NDST3, CISH, SLCO1C1, SOCS2, SERPINC1, CGNL1, and TDRD5.

30. A method, comprising:
contacting a kidney cell with an agent that modifies (a) expression of a gene selected from any one of the genes listed in Table 1, or (b) activity of a product encoded by a gene selected from any one of the genes listed in Table 1.

31. (canceled)

32. A method, comprising:
contacting a kidney cell with an agent that modifies expression of, or modifies activity of a product encoded by, a pathway gene upstream or downstream from a gene selected from NDST3, CISH, SLCO1C1, SOCS2, SERPINC1, CGNL1, and TDRD5.

33. (canceled)
